SEQUENCE LISTING (48 sequences)

SEQ ID NO: 1 - protein, 805 aa, Homo sapiens [sequence omitted]

SEQ ID NO: 2 - protein, 37 aa, Artificial sequence
  Synthetic sequence
STIEEQAKTFLDKFNHEAEDLFYQSSLASWNYNTNIT

SEQ ID NO: 3 - protein, 21 aa, Artificial sequence
  Synthetic sequence
EEQAKTFLDKFNHEAEDLFYQ

SEQ ID NO: 4 - protein, 45 aa, Artificial sequence
  Synthetic sequence
STIEEQAKTFLDKFNHEAEDLFYQSSLASWNYNTNITRRRRRRRR

SEQ ID NO: 5 - protein, 29 aa, Artificial sequence
  Synthetic sequence
EEQAKTFLDKFNHEAEDLFYQRRRRRRRR

SEQ ID NO: 6 - protein, 38 aa, Artificial Sequence
  Synthetic sequence
  misc_feature (38)..(38): Xaa can be any naturally occurring amino acid
STIEEQAKTFLDKFNHEAEDLFYQSSLASWNYNTNITX

SEQ ID NO: 7 - protein, 39 aa, Artificial sequence
  Synthetic sequence
  misc_feature (38)..(39): Xaa can be any naturally occurring amino acid
STIEEQAKTFLDKFNHEAEDLFYQSSLASWNYNTNITXX

SEQ ID NO: 8 - protein, 40 aa, Artificial sequence
  synthetic sequence
  misc_feature (38)..(40): Xaa can be any naturally occurring amino acid
STIEEQAKTFLDKFNHEAEDLFYQSSLASWNYNTNITXXX

SEQ ID NO: 9 - protein, 41 aa, Artificial sequence
  synthetic sequence
  misc_feature (38)..(41): Xaa can be any naturally occurring amino acid
STIEEQAKTFLDKFNHEAEDLFYQSSLASWNYNTNITXXXX

SEQ ID NO: 10 - protein, 42 aa, Artificial sequence
  synthetic sequence
  misc_feature (38)..(42): Xaa can be any naturally occurring amino acid
STIEEQAKTFLDKFNHEAEDLFYQSSLASWNYNTNITXXXXX

SEQ ID NO: 11 - protein, 43 aa, Artificial sequence
  synthetic sequence
  misc_feature (38)..(43): Xaa can be any naturally occurring amino acid
STIEEQAKTFLDKFNHEAEDLFYQSSLASWNYNTNITXXXXXX

SEQ ID NO: 12 - protein, 44 aa, Artificial sequence
  Synthetic sequence
  misc_feature (38)..(44): Xaa can be any naturally occurring amino acid
STIEEQAKTFLDKFNHEAEDLFYQSSLASWNYNTNITXXXXXXX

SEQ ID NO: 13 - protein, 46 aa, Artificial sequence
  Synthetic sequence
  misc_feature (38)..(46): Xaa can be any naturally occurring amino acid
STIEEQAKTFLDKFNHEAEDLFYQSSLASWNYNTNITXXXXXXXXX

SEQ ID NO: 14 - protein, 47 aa, Artificial sequence
  Synthetic sequence
  misc_feature (38)..(47): Xaa can be any naturally occurring amino acid
STIEEQAKTFLDKFNHEAEDLFYQSSLASWNYNTNITXXXXXXXXXX

SEQ ID NO: 15 - protein, 48 aa, Artificial sequence
  synthetic sequence
  misc_feature (38)..(48): Xaa can be any naturally occurring amino acid
STIEEQAKTFLDKFNHEAEDLFYQSSLASWNYNTNITXXXXXXXXXXX

SEQ ID NO: 16 - protein, 22 aa, Artificial sequence
  Synthetic sequence
  misc_feature (22)..(22): Xaa can be any naturally occurring amino acid
EEQAKTFLDKFNHEAEDLFYQX

SEQ ID NO: 17 - protein, 23 aa, Artificial sequence
  Synthetic sequence
  misc_feature (22)..(23): Xaa can be any naturally occurring amino acid
EEQAKTFLDKFNHEAEDLFYQXX

SEQ ID NO: 18 - protein, 24 aa, Artificial sequence
  Synthetic sequence
  misc_feature (22)..(24): Xaa can be any naturally occurring amino acid
EEQAKTFLDKFNHEAEDLFYQXXX

SEQ ID NO: 19 - protein, 25 aa, Artificial sequence
  Synthetic sequence
  misc_feature (22)..(25): Xaa can be any naturally occurring amino acid
EEQAKTFLDKFNHEAEDLFYQXXXX

SEQ ID NO: 20 - protein, 26 aa, Artificial sequence
  synthetic sequence
  misc_feature (22)..(26): Xaa can be any naturally occurring amino acid
EEQAKTFLDKFNHEAEDLFYQXXXXX

SEQ ID NO: 21 - protein, 27 aa, Artificial sequence
  synthetic sequence
  misc_feature (22)..(27): Xaa can be any naturally occurring amino acid
EEQAKTFLDKFNHEAEDLFYQXXXXXX

SEQ ID NO: 22 - protein, 28 aa, Artificial sequence
  synthetic sequence
  misc_feature (22)..(28): Xaa can be any naturally occurring amino acid
EEQAKTFLDKFNHEAEDLFYQXXXXXXX

SEQ ID NO: 23 - protein, 29 aa, Artificial sequence
  synthetic sequence
  misc_feature (22)..(29): Xaa can be any naturally occurring amino acid
EEQAKTFLDKFNHEAEDLFYQXXXXXXXX

SEQ ID NO: 24 - protein, 30 aa, Artificial sequence
  synthetic sequence
  misc_feature (22)..(30): Xaa can be any naturally occurring amino acid
EEQAKTFLDKFNHEAEDLFYQXXXXXXXXX

SEQ ID NO: 25 - protein, 31 aa, Artificial sequence
  synthetic sequence
  misc_feature (22)..(31): Xaa can be any naturally occurring amino acid
EEQAKTFLDKFNHEAEDLFYQXXXXXXXXXX

SEQ ID NO: 26 - protein, 32 aa, Artificial sequence
  synthetic sequence
  misc_feature (22)..(32): Xaa can be any naturally occurring amino acid
EEQAKTFLDKFNHEAEDLFYQXXXXXXXXXXX

SEQ ID NO: 27 - protein, 24 aa, Artificial sequence
  Synthetic sequence
EEQAKTFLDKFNHEAEDLFYQSSL

SEQ ID NO: 28 - protein, 25 aa, Artificial sequence
  Synthetic sequence
  misc_feature (25)..(25): Xaa can be any naturally occurring amino acid
EEQAKTFLDKFNHEAEDLFYQSSLX

SEQ ID NO: 29 - protein, 26 aa, Artificial sequence
  Synthetic sequence
  misc_feature (25)..(26): Xaa can be any naturally occurring amino acid
EEQAKTFLDKFNHEAEDLFYQSSLXX

SEQ ID NO: 30 - protein, 27 aa, Artificial sequence
  Synthetic sequence
  misc_feature (25)..(27): Xaa can be any naturally occurring amino acid
EEQAKTFLDKFNHEAEDLFYQSSLXXX

SEQ ID NO: 31 - protein, 28 aa, Artificial sequence
  Synthetic sequence
  misc_feature (25)..(28): Xaa can be any naturally occurring amino acid
EEQAKTFLDKFNHEAEDLFYQSSLXXXX

SEQ ID NO: 32 - protein, 29 aa, Artificial sequence
  Synthetic sequence
  misc_feature (25)..(29): Xaa can be any naturally occurring amino acid
EEQAKTFLDKFNHEAEDLFYQSSLXXXXX

SEQ ID NO: 33 - protein, 30 aa, Artificial sequence
  Synthetic sequence
  misc_feature (25)..(30): Xaa can be any naturally occurring amino acid
EEQAKTFLDKFNHEAEDLFYQSSLXXXXXX

SEQ ID NO: 34 - protein, 31 aa, Artificial sequence
  Synthetic sequence
  misc_feature (25)..(31): Xaa can be any naturally occurring amino acid
EEQAKTFLDKFNHEAEDLFYQSSLXXXXXXX

SEQ ID NO: 35 - protein, 32 aa, Artificial sequence
  Synthetic sequence
  misc_feature (25)..(32): Xaa can be any naturally occurring amino acid
EEQAKTFLDKFNHEAEDLFYQSSLXXXXXXXX

SEQ ID NO: 36 - protein, 33 aa, Artificial sequence
  Synthetic sequence
  misc_feature (25)..(33): Xaa can be any naturally occurring amino acid
EEQAKTFLDKFNHEAEDLFYQSSLXXXXXXXXX

SEQ ID NO: 37 - protein, 34 aa, Artificial sequence
  Synthetic sequence
  misc_feature (25)..(34): Xaa can be any naturally occurring amino acid
EEQAKTFLDKFNHEAEDLFYQSSLXXXXXXXXXX

SEQ ID NO: 38 - protein, 35 aa, Artificial sequence
  Synthetic sequence
  misc_feature (25)..(35): Xaa can be any naturally occurring amino acid
EEQAKTFLDKFNHEAEDLFYQSSLXXXXXXXXXXX

SEQ ID NO: 39 - protein, 25 aa, Artificial sequence
  Synthetic sequence
EEQAKTFLDKFNHEAEDLFYQSSLR

SEQ ID NO: 40 - protein, 26 aa, Artificial sequence
  Synthetic sequence
EEQAKTFLDKFNHEAEDLFYQSSLRR

SEQ ID NO: 41 - protein, 27 aa, Artificial sequence
  Synthetic sequence
EEQAKTFLDKFNHEAEDLFYQSSLRRR

SEQ ID NO: 42 - protein, 28 aa, Artificial sequence
  Synthtetic sequence
EEQAKTFLDKFNHEAEDLFYQSSLRRRR

SEQ ID NO: 43 - protein, 29 aa, Artificial sequence
  Synthetic sequence
EEQAKTFLDKFNHEAEDLFYQSSLRRRRR

SEQ ID NO: 44 - protein, 30 aa, Artificial Sequence
  Synthetic sequence
EEQAKTFLDKFNHEAEDLFYQSSLRRRRRR

SEQ ID NO: 45 - protein, 30 aa, Artificial Sequence
  Synthetic sequence
EEQAKTFLDKFNHEAEDLFYQSSLHHHHHH

SEQ ID NO: 46 - protein, 32 aa, Artificial Sequence
  Synthetic sequence
EEQAKTFLDKFNHEAEDLFYQSSLRRHHHHHH

SEQ ID NO: 47 - protein, 31 aa, Artificial Sequence
  synthetic sequence
EEQAKTFLDKFNHEAEDLFYQSSLRRHHHHH

SEQ ID NO: 48 - protein, 30 aa, Artificial Sequence
  Synthetic sequence
EEQAKTFLDKFNHEAEDLFYQSSLRRHHHH

(19) United States
(12) Patent Application Publication
(10) Pub. No.: US 2023/0235308 A1
(43) Pub. Date: Jul. 27, 2023

(54) METHODS, COMPOSITIONS, AND PROPHYLACTICS FOR TREATING, AMELIORATING, OR PREVENTING CORONAVIRUS DISEASE (COVID-19)

(71) Applicant: The Research Foundation for the State University of New York, Albany, NY (US)

(72) Inventors: Matthew Pincus, Brooklyn, NY (US); Steven Evans, New York, NY (US); Fred Friedman, Silver Spring, MD (US)

(21) Appl. No.: 18/011,430

(22) PCT Filed: Jun. 18, 2021
(86) PCT No.: PCT/US2021/038107

§ 371 Date: Dec. 19, 2022

Related U.S. Application Data
(60) Provisional application No. 63/041,686, filed on Jun. 19, 2020.

(51) Int. Cl. C12N 9/48; A61P 31/14

(52) U.S. Cl. CPC C12N 9/485; C12Y 304/17023; A61P 31/14

(57) ABSTRACT

Disclosed are the methods and compositions for treating, ameliorating, or preventing COVID-19 or conditions associated with SARS-CoV-2 infection, and, also for reversing the damage caused by SARS-CoV-2 infection. Pharmaceutically acceptable compositions including spike protein binding partners, and optionally personal protective equipment included spike protein binding partners, are also disclosed.

### CROSS-REFERENCE TO RELATED APPLICATIONS

The present disclosure claims priority or the benefit under 35 U.S.C. § 119 of U.S. provisional application No. 63/041,686 filed Jun. 19, 2020, the contents of which are fully incorporated herein by reference.

### REFERENCE TO A SEQUENCE LISTING

This application contains a Sequence Listing in computer readable form, which is incorporated herein by reference.

### FIELD OF THE INVENTION

The present disclosure relates to methods, compositions such as peptide drugs and prophylactics such as personal protective equipment (PPE) for treating, ameliorating, or preventing COVID-19.

### BACKGROUND

The World Health Organization (WHO) declared infection by the Severe Acute Respiratory Syndrome Coronavirus 2 (SARS-CoV-2), as a pandemic, and termed the related disease as coronavirus disease 2019 (COVID-19). Although a large percentage of persons infected with the virus experience mild to moderate respiratory, gastrointestinal, cardiovascular, or other discomforts without requiring medical care, infected persons with underlying medical problems, comorbidities, such as diabetes, cardiovascular disease, chronic respiratory disease or cancer are more likely to develop serious illness and/or die from COVID-19 or related secondary infections.

The coronavirus includes four protein types including spike, envelope, membrane, and capsid proteins. The spike proteins protrude away from the virus membrane surface and collectively form a crown-like structure. When contacting and infecting humans, the spike proteins interact with human host cells by binding to angiotensin converting enzyme-2 (ACE-2) that is on the surface of these cells, and the spike proteins are cleaved into two separate proteins e.g. S1 and S2. After contact and cleavage, the S1 protein facilitates binding to the angiotensin 2 receptor on the cell surface and the S2 protein mediates membrane fusion.

ACE2 is almost all α-helical, and spike proteins predominantly bind to its α-helix 1 domain, including residues 19-55. Transmission vectors of the virus, and variants thereof, are under heavy investigation at this time, and it is suspected the virus may be aerosolized and spread through saliva or discharge from the nose and throat when an infected person coughs or sneezes. While social distancing appears to reduce the number of individuals infected at one time, there is presently no consensus on appropriate therapies to treat, ameliorate or prevent the transmission of SARS-CoV-2 to a human host resulting in COVID-19.

What is needed are compositions and methods of treating, ameliorating, or preventing COVID-19. Moreover, PPE configured to contain SARS-CoV-2 and prevent COVID-19 is also needed.

### SUMMARY

The present disclosure relates to compositions, and methods for treating, ameliorating, or preventing COVID-19. Personal protective equipment (PPE) for the containment of or entrapment of SARS-CoV-2 and preventing COVID-19 is also described. Carriers or substrates that may bind SARS-CoV-2, or variants thereof, are also described. Moreover, a device for pulmonary delivery of one or more spike protein binding partners of the present disclosure is described.

In embodiments, the present disclosure relates to a recombinant variant of a parent angiotensin-converting enzyme 2 including: a plurality of deletions of amino acid residues corresponding to amino acid residues M1 to Q18 and E56 to F805 using SEQ ID NO: 1 for numbering; and wherein the variant has increased stability, spike protein binding activity, and/or COVID-19 treatment performance benefit compared to the parent angiotensin-converting enzyme 2 or a reference angiotensin-converting enzyme 2 differing from the variant angiotensin-converting enzyme 2 only by the absence of the deletions.

In some embodiments the present disclosure relates to a synthetic amino acid sequence, including, or consisting of SEQ ID NOS: 2-48. In some embodiments, the synthetic amino acids sequences or peptides include one or more His-Tags fused to the C-terminus (carboxy-terminus), or N-terminus (amino-terminus).

In some embodiments, the present disclosure relates to a complementary deoxynucleotide (cDNA) sequence encoding an amino acid sequence having at least at least 90%, at least 95%, or at least 99% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48. In some embodiments, the cDNA of the present disclosure includes DNA that encodes one or more His-Tags of the present disclosure. In embodiments, the one or more His-Tags are fused to one or more polypeptides of the present disclosure.

In some embodiments, the present disclosure relates to a composition including the variant angiotensin-converting enzyme 2 described herein, or a synthetic amino acid sequence, including, or consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48. In some embodiments, the variant angiotensin-converting enzyme 2 described herein, or a synthetic amino acid sequence described herein further includes one or more His-Tags of the present disclosure.

In some embodiments, the present disclosure relates to a method of treating, ameliorating, or preventing one or more symptoms of COVID-19 in a subject having one or more COVID-19 symptoms, comprising: administering a therapeutically effective amount of spike protein binding partner to a subject in need thereof. In embodiments, the spike protein binding partner is an ACE2 variant which serves as a spike protein binding partner.

In some embodiments, the present disclosure relates to an article of personal protection equipment, including: a plurality of polypeptides having at least 90%, at least 95%, or at least 99% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48, or combinations thereof disposed within or upon the surface of the personal protection equipment, wherein the personal protection equipment is configured to contain SARS-CoV-2, or a variant thereof, when SARS-CoV-2 or a variant thereof contacts the personal protection equipment.

In embodiments, the present disclosure relates to a bead including: a substrate; and one or more spike protein binding partners. In embodiments, the one or more spike protein binding partners comprise a His-Tag, wherein the His-Tag is bound to the substrate. In embodiments, the substrate comprises metal such as nickel. In embodiments, the bead is characterized as a nanobead. In embodiments, two or more, a plurality, of beads of the present disclosure are provided.

The illustrative aspects of the present disclosure are designed to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 depicts is a cross-sectional side-view of a drug delivery device in accordance with the present disclosure.

FIG. 2 depicts a partial cross-section frontal-view of a respirator in accordance with the present disclosure.

FIG. 3 depicts a partial cross-section frontal-view of a respirator in accordance with the present disclosure.

FIG. 4 depicts a composition embodiment of the present disclosure.

FIG. 5A depicts a composition embodiment of the present disclosure.

FIG. 5B depicts a composition embodiment of the present disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The compositions and methods described herein include administering a therapeutically effective amount of spike protein binding partner to a subject in need thereof. In embodiments, the methods of the present disclosure provide a prompt and effective treatment to block SARS-CoV-2 (or a variant thereof) infection. In embodiments, the compositions and methods of the present disclosure prevent the SARS-CoV-2 virus, or variants thereof, from binding to ACE2 cell receptors and entering mammalian cells by receptor-mediated endocytosis.

Without wishing to be bound by any theory, it is believed that SARS-CoV-2, or variant thereof, attaches to mammalian or human ACE2 cell receptors by using a receptor-binding domain (RBD) of the “spike” protein on an envelope protein coat of the SARS-CoV-2 virus. The ACE2 cell receptor is angiotensin converting enzyme 2, an enzyme that hydrolyzes angiotensin II resulting in inactivation of angiotensin II. ACE2 binds to human cells through a hydrophobic sequence near the carboxyl terminal end of the enzyme, with an adjacent sequence that is the only intracellular segment of this protein containing phosphorylation sites. ACE2 is expressed on the vascular endothelial cells of lung, kidney and heart. In vivo, ACE2 is subjected to a cleavage process that releases ACE2 from the cell membrane resulting in ACE2 secretion into the blood stream. Accordingly, ACE2 is immunologically tolerated. Both cell bound ACE2 and free or circulating ACE2 are enzymatically active and contain a coronavirus binding site.

The ACE 2 receptors occur principally in type II pneumocytes. Viral (SARS-CoV-2 or variants thereof) penetration of the pneumocytes may lead to severe if not fatal lung infection, and/or other infections such as in the gastrointestinal tract (viral penetration causing severe GI symptoms including diarrhea). Benefits of the embodiments of the present disclosure prevent, reduce, ameliorate and/or cure SARS-CoV-2 infections and/or COVID-19.

Further, as noted above, ACE2 (SEQ ID NO: 1) is mostly an α-helical protein including five long helical segments. Both spike proteins S1 and S2 principally bind in an almost identical manner to one α-helical domain of ACE2, the α-helix 1, or al, domain on the amino terminal end of the protein including amino acid residues 19-55. This region includes the peptide residues that are the major contacts between the viral RBD and ACE2. It has been observed that the α-helical domain of ACE2 appears to interact minimally with the remainder of the ACE2 protein and is exposed on the surface of the protein. It has also been observed that the structure of the al helical domain appears to be unchanged whether bound to the RBD of the spike protein, or not bound to the RBD of the spike protein. In embodiments, superposition of the al domain (residues 22-45) of Covid-2-bound ACE2 and free ACE2 results in a root mean square deviation (RMSD)Å of only 0.58 Å, showing that the conformation of the α1 domain is fixed and stable.

Further, the isolated peptides such as segment 19-55 of ACE2 adopt the α-helical conformation which is configured to bind to the RBD of SARS-CoV-2, and the isolated peptides of the disclosure are beneficial as competitive inhibitors of SARS-CoV-2 binding to the SARS-CoV-2 native ACE2 receptor on cells or unbound. In embodiments, the isolated peptides of the disclosure are provided to a patient in need thereof in an amount sufficient to competitively inhibit SARS-CoV-2 binding to the SARS-CoV-2 native ACE2 receptor on cells or unbound. Thus, the polypeptides of the present disclosure are useful for treatment for coronavirus infection.

It has further been observed that since the viral-binding peptides of the present disclosure are immunologically tolerated and maintain a functional conformation independently of the remainder of the protein and do not change conformation when bound to the RBD of SARS-CoV-2, peptides from the al domain of ACE2 and variants thereof, such as for example peptides that include peptides from the al domain of ACE2, are suitable as agents that bind the coronavirus in an infected individual and inhibit coronavirus binding to coronavirus cellular receptor(s), and are thus configured for neutralizing SARS-CoV-2 as an infectious agent. Further, the virus-peptide complexes formed in accordance with the methods of the present disclosure are excreted or phagocytosed such that the virus is eliminated from the human body.

It has also been observed that a major impediment in using a protein-derived peptide to competitively inhibit protein binding to ACE2 receptor, is that the isolated peptide may generally not be expected to adopt a suitable or functional conformation within the intact protein. This occurs because isolated peptide is problematically not subjected to the structural constraints experienced when juxtaposed to other protein elements, resulting in multiple bonding interactions with distal residues of the protein. Indeed, isolated peptides such as fragments of ACE 2 (SEQ ID NO:1) are unlikely to exist in a single conformation but are rather thought of adopting a multiplicity of low energy conformations. This is an often-intractable problem, whose resolution requires skilled insight and knowledge of the factors which govern protein structure. The present disclosure beneficently provides polypeptides of the present disclosure including a nativelike alpha helical structure for the isolated peptide of the present disclosure such as those corresponding to residues 19-55 of ACE2. In some embodiments, the present disclosure beneficently provides polypeptides of the present disclosure including a nativelike alpha helical structure for the isolated peptide of the present disclosure such as those corresponding to residues 22-45 of ACE2 or including residues 22-45 of SEQ ID NO: 1.

In embodiments, the present disclosure relates to compositions and methods for application of one or more spike protein binding partners to subjects in need thereof. The methods include administering a predetermined amount of one or more spike protein binding partners to a subject in need thereof such as a therapeutic effective amount. A treatment in accordance with the present disclosure includes treating subjects in need thereof with one or more spike protein binding partners to treat, ameliorate, or prevent COVID-19, or one or more symptoms of COVID-19 in a subject such as lung or gastrointestinal illness induced by SARS-CoV-2 or a variant thereof. Further, compositions and methods of the present disclosure counteract SARS-CoV-2 exposures, and prophylactically prevent SARS-CoV-2 infection.

Spike protein binding partner therapy as described herein is beneficial in that it is a cost-effective treatment option. As such, in embodiments, the present disclosure provides the benefit of blocking the SARS-CoV-2 virus, or variants thereof, from binding with one or more human receptors and/or causing human infection. Benefits of embodiments of the present disclosure include subject recovery from SARS-CoV-2 infection and avoidance of infection. Further, spike protein binding partners beneficially act as protective agents and ameliorate COVID-19 disease emanating from SARS-CoV-2, or a variant thereof. Described herein are spike protein binding partners or competitive inhibitors of SARS-CoV-2 or SARS-CoV-2 variants. Further described herein are synthetic polypeptides, and variants configured or provided as spike protein binding partners. Also described herein are methods for synthesizing such spike protein binding partners or competitive inhibitors, and methods for using such spike protein binding partners as competitive inhibitors in the treatment of diseases (including diseases wherein competitive inhibition of SARS-CoV-2 provides therapeutic benefit to a patient having COVID-19). Further described are pharmaceutical formulations that include one or more competitive inhibitors of SARS-CoV-2, e.g. a pharmaceutically acceptable formulation for contacting pulmonary tissues or organs, and PPE including one or more competitive inhibitors of SARS-CoV-2, or a variant thereof, or one or more spike protein binding partners configured to fix SARS-CoV-2 to the PPE. In embodiments, a composition is provided where one or more spike protein binding partners include a His-Tag and/or are bound to a substrate such as a metal substrate.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the term “ACE2” refers to Angiotensin II converting enzyme (ACE2, EC 3.4.17.23). ACE2 is a protein that catalyzes the cleavage of angiotensin I into angiotensin 1-9, and angiotensin II into the vasodilator angiotensin 1-7. In addition, the encoded protein is a functional receptor for the spike glycoprotein of the human coronavirus HCoV-NL63 and the human severe acute respiratory syndrome coronaviruses, SARS-CoV and SARS-CoV-2 (COVID-19 virus). ACE2 converting enzyme activity may be measured using, inter alia, fluorometric activity assay kits and by other known methods such as those described in Measurement of Angiotensin Converting Enzyme 2 Activity in Biological Fluid (ACE2), Methods Mol Biol. 2017; 1527:101-115.

In non-limiting embodiments, polypeptides of the present disclosure have at least 50%, at least 80%, at least 70%, at least 80%, at least 90%, at least 95%, at least 99% or at least 100% of the binding affinity to SARS-CoV-2 spike protein of the mature polypeptide of SEQ ID NO: 1. In non-limiting embodiments, polypeptides of the present disclosure have at least 50%, at least 80%, at least 70%, at least 80%, at least 90%, at least 95%, at least 99% or at least 100% of the binding affinity to SARS-CoV-2 variant spike protein.

As used herein the term “cDNA” refers to a DNA molecule that can be prepared by reverse transcription from an RNA molecule obtained from a eukaryotic or prokaryotic cell, a virus, or from a sample solution. In embodiments, cDNA lacks introns or intron sequences that may be present in corresponding genomic DNA. In embodiments, cDNA may refer to a nucleotide sequence that corresponds to the nucleotide sequence of an RNA from which it is derived. In embodiments, cDNA refers to a double-stranded DNA that is complementary to and derived from mRNA.

As used herein the term “coding sequence” means a polynucleotide, which directly specifies the amino acid sequence of a polypeptide. In embodiments, boundaries of the coding sequence may be determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

As used herein the term “competitive inhibitor” refers to one or more substances that binds to or blocks another substance from participating in a reaction. For example, spike protein binding partners of the present disclosure bind to coronavirus spike protein and block it from binding to the cellular receptor ACE2.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

As used herein the terms “drug,” “drug substance,” “active pharmaceutical ingredient,” and the like, refer to a compound (e.g., spike protein binding partner) that may be used for treating a subject in need of treatment.

As used herein the term “excipient” or “adjuvant” refers to any inert substance.

As used herein the terms “drug product,” “pharmaceutical dosage form,” “dosage form,” “final dosage form” and the like, refer to a pharmaceutical composition that is administered to a subject in need of treatment and generally may be in the form of inhalers, tablets, capsules, sachets containing powder or granules, liquid solutions or suspensions, patches, and the like.

As used herein the term “fragment” means a polypeptide having one or more (e.g., several) amino acids absent from the amino and/or carboxyl terminus of a mature polypeptide or domain. In embodiments, a fragment is able to bind to the SARS-CoV-2 spike protein or to SARS-CoV-2 variant spike protein. In embodiments, a fragment contains at least 1% to 30%, at least 2% to 20% or about 2 to 10% of the number of amino acids of the mature polypeptide of SEQ ID NO: 1. In embodiments, a fragment includes amino acids corresponding to positions 22-45 (using SEQ ID NO: 1 for numbering). In embodiments, a fragment contains at least 70% to 99%, at least 90% to 99% or about 95 to 99% of the number of amino acids of the mature polypeptide of SEQ ID NOS: 2-48.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The term “isolated” means a substance in a form or environment that does not occur in nature. Non-limiting examples of isolated substances include (1) any non-naturally occurring substance, (2) any substance such as a variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The term “mature polypeptide” means a polypeptide in its final form following translation and any post-translational modifications, such as N-terminal processing, C-terminal truncation, glycosylation, etc.

The term “nucleotide” refers to a ribonucleotide or a deoxyribonucleotide or modified form thereof, as well as an analog thereof.

As used herein, the term “nucleic acid molecule” refers to any molecule containing multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

As used here, the term “SARS-CoV-2” refers to virus classified within the genus Betacoronavirus (subgenus Sarbecovirus) in the family Coronaviridae (subfamily Orthocoronavirinae), a family of single-strand positive-sense RNA viruses. In embodiments, the term “SARS-CoV-2” includes variants of SARS-CoV-2.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

It would be understood that, when calculating sequence identity between a DNA sequence and an RNA sequence. T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

As used herein the term “solvate” describes a molecular complex including the drug substance (e.g., spike protein binding partner) and a stoichiometric or non-stoichiometric amount of one or more pharmaceutically acceptable solvent molecules.

The term “substantially purified,” as used herein, refers to a component of interest that may be substantially or essentially free of other components which normally accompany or interact with the component of interest prior to purification. In embodiments, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. In embodiments, a component of interest includes a virus-of-interest, such as SARS-CoV-2 or a variant thereof.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology. “Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

The term “hydrate” describes a solvate including the drug substance and a stoichiometric or non-stoichiometric amount of water.

As used herein the term “pharmaceutically acceptable” substances refers to those substances, such as e.g., spike protein binding partner, which are within the scope of sound medical judgment suitable for use in contact with the tissues of subjects without undue toxicity, irritation, allergic response, and the like, and effective for their intended use.

As used herein the term “pharmaceutical composition” refers to the combination of one or more drug substances such as e.g., spike protein binding partner and one or more excipients and one or more pharmaceutically acceptable vehicles with which the one or more spike protein binding partner is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids; and salts formed when an acidic proton present in the parent compound is replaced by a metal ion, for example, an alkali metal ion, an alkaline earth ion, or an aluminum ion. In embodiments, spike protein binding partners of the present disclosure may be in a pharmaceutically acceptable salt form.

As used herein the term “pharmaceutically acceptable vehicle” refers to a diluent, adjuvant, excipient or carrier with which a compound, such as e.g., spike protein binding partner, is administered.

As used herein the term “prevent”, “preventing” and “prevention” of COVID-19 means (1) reducing the risk of a patient who is not experiencing symptoms of SARS-CoV-2 infection from developing COVID-19, or (2) reducing the frequency of, the severity of, or a complete elimination of COVID-19 symptoms already being experienced by a subject.

The term “prophylactically effective amount,” as used herein, refers to that amount of a composition, such as e.g., spike protein binding partner, applied to a patient or personal protective equipment which will relieve to some extent one or more of the symptoms of a disease, likelihood of becoming diseased, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

As used herein the term “subject” includes humans, animals or mammals. The terms “subject” and “patient” may be used interchangeably herein.

As used herein, the term “selective binding compound” refers to a compound that selectively binds to any portion of one or more target proteins.

As used herein, “spike protein binding partner” refers to a compound that selectively binds to any portion of one or more spike proteins or fragments thereof of SARS-CoV-2, or a variant thereof, such as proteins e.g. S1 and S2. In embodiments, spike protein binding partner is a selective binding compound.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

As used herein, the term “target protein” refers to a molecule or a portion of a protein capable of being bound by a selective binding compound. In certain embodiments, a target protein is SARS-CoV-2 (including variants thereof) spike protein or fragments thereof such as S1 and/or S2.

As used herein the term “therapeutically effective amount” means the amount of a compound that, when administered to a subject for treating or preventing SARS-CoV-2 infection, is sufficient to effect such treatment or prevention of COVID-19 and related symptoms. A “therapeutically effective amount” can vary depending, for example, on the compound, the severity of the SARS-CoV-2 infection, the etiology of the SARS-CoV-2 infection, the age of the subject to be treated, comorbidities of the subject to be treated, existing health conditions of the subject, and/or the weight of the subject to be treated. A “therapeutically effective amount” is an amount sufficient to alter the subjects' natural state.

As used herein the term “treat”, “treating” and “treatment” of COVID-19 means an intervention for reducing the frequency of symptoms of COVID 19, eliminating the symptoms of COVID-19, avoiding or arresting the development of symptoms of COVID-19, ameliorating or curing an existing or undesirable symptom caused by SARS-CoV-2, and/or reducing the severity of symptoms of COVID-19.

In embodiment the term “variant” means a polypeptide SARS-CoV-2 spike protein binding partner including an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-10 amino acids, adjacent to the amino acid occupying a position. In some embodiments, the term “variant” refers to a SARS-CoV-2 virus variant.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS

The compositions, and methods described herein include administering to a subject in need a composition containing a therapeutically effective amount of one or more SARS-CoV-2 inhibitor or SARS-CoV-2 variant inhibitor compounds described herein.

In embodiments, the term SARS-CoV-2 includes virus from a reference strain of SARS-CoV-2 referred to as Wuhan-Hu-1 (Gen Bank accession MN908947) or ‘the original Wuhan strain’, sampled from a patient in Wuhan, China, on 26 Dec. 2019. As used herein the term SARS-CoV-2 includes variants of SARS-CoV-2. Although nomenclature for SARS-CoV-2 variants is not uniform one of ordinary skill in the art understands that established nomenclature for naming and tracking SARS-CoV-2 genetic lineages by GISAID, Nextstrain, and Pango are currently in use. Recently, the World Health Organization (WHO) recommended labeling SARS-CoV-2 variants using letters of the Greek Alphabet to refer to variants, such as variants of concern and variants of interest (See e.g., the world wide web at www.who.int/en/activities/tracking-SARS-CoV-2-variants/.). Accordingly, non-limiting examples of SARS-CoV-2 variants include variants of concern, or variants having an observed increase in transmissibility or detrimental change in the COVID 19 epidemiology, increase in virulence or change in clinical presentation, or decrease in effectiveness of preventative measures such as social distancing and vaccination. Non-limiting examples of current variants-of-concern include the Alpha, Beta, Gamma, and Delta (WHO labelled variants), or B.1.17 (documented first in the U.K.), B.1.351 (documented first in South Africa), P.1 (documented first in Brazil), B.1.617.2 (documented first in India) (Pango lineage), respectively. Variants of interest include SARS-CoV-2 isolate, that when compared to a reference isolate, its genome has mutations with established or suspected phenotypic implications. Non-limiting examples of current variants of interest include Epsilon, Zeta, Eta, Theta, Iota, and Kappa (WHO labelled variants), or B.1427/61.429, P2, B.1.525, P3, B.1.526, B.1.6171.1 (Pango lineage), respectively. Variants also include natural or manmade variants of SARS-CoV-2 that have not yet formed, thus are not yet identified or named.

In some embodiments, SARS-CoV-2 refers to Wuhan-Hu-1 (GenBank accession MN908947), sampled from a patient in Wuhan, China, on 26 Dec. 2019 (See e.g., Wu F, Zhao S, Yu B, Chen Y-M, Wang W, Song Z-G et al. A new coronavirus associated with human respiratory disease in China. Nature. 2020; 579:265-9. doi: 10.1038/s41586-020-2008-3). That genome is 29 903 nucleotides (nt) in length and includes a gene order of similar structure to that seen in other coronaviruses: 5′-replicase ORF1ab-S-E-M-N-3′. The predicted replicase ORF1ab gene of Wuhan-Hu-1 is 21 291 nt in length. The ORF1ab polyprotein is predicted to be cleaved into 16 nonstructural proteins. ORF1ab is followed by a number of downstream open reading frames (ORFs). These include the predicted S (spike), ORF3a, E (envelope), M (membrane) and N (nucleocapsid) genes of lengths 3822, 828, 228, 669 and 1260 nt, respectively. Like SARS-CoV, Wuhan-Hu-1 also contains a predicted ORF8 gene (366 nt in length) located between the M and N genes. Finally, the 5′ and 3′ terminal sequences of Wuhan-Hu-1 are also typical of betacoronaviruses and have lengths of 265 nt and 229 nt, respectively. See e.g., Genomic sequencing of SARS-CoV-2: a guide to implementation for maximum impact on public health, 8 Jan. 2021, COVID-19: Laboratory and diagnosis available on the world wide web at www.who.int/publications/i/item/9789240018440.

Without being bound by theory, the role played by SARS-CoV-2 spike protein or SARS-CoV-2 variant spike protein in contacting and infecting cells, suggests that spike protein binding partners are useful as SARS-CoV-2 inhibitors and for reducing the risk of or treating a variety of symptoms of COVID-19.

The present disclosure relates to a method of treating, ameliorating, or preventing one or more symptoms of COVID-19 in a subject having one or more COVID-19 symptoms, including: administering a therapeutically effective amount of spike protein binding partner to a subject in need thereof. In embodiments, the present disclosure uses spike protein binding partner, to beneficially counteract against the SARS-CoV-2 virus. In embodiments, the compositions and methods of the present disclosure beneficially treat, ameliorate, prevent, or reduce symptoms associated with and/or caused by SARS-CoV-2 virus. The compositions and methods of the present disclosure beneficially provide a drug to treat COVID-19 throughout the body and methods of treating, ameliorating, or preventing one or more symptoms or conditions associated with or caused by SARS-CoV-2 virus and/or reversing the damage caused by prolonged or acute SARS-CoV-2 virus exposure. Further, prophylaxis for subjects such as first responders including health care workers that continue to face SARS-CoV-2 virus exposures are provided along with therapies for protection.

In embodiments, the present disclosure combats SARS-CoV-2 virus, or variants thereof, in subjects in need thereof using spike protein binding partners such as functional derivatives or fragments of ACE2 enzyme (SEQ ID NO:1). In embodiments, spike protein binding partners are useful for counteracting SARS-CoV-2 virus exposures, and for recovering losses in bodily function induced by SARS-CoV-2 virus infection. In embodiments, spike protein binding partner treatment in accordance with the present disclosure ameliorates symptoms to levels comparable with subjects that were not exposed to any SARS-CoV-2. In embodiments, spike protein binding partner treatment in accordance with the present disclosure ameliorates symptoms such as pneumonia, or gastrointestinal distress, and blood clotting to levels at least 10%, at least 25%, at least 50%, or between 10% and 95% improved compared to the subject's initial presentation for treatment. Moreover, spike protein binding partner treatment in accordance with the present disclosure can be tailored based on level of exposure or comorbidity, to maximize efficacy. To further increase efficacy, spike protein binding partner treatment in accordance with the present disclosure can be manufactured as pharmaceutical dosage forms such as inhalers to meet the specific needs of patients based on their clinical profiles and unique symptomology.

In embodiments, methods for using spike protein binding partner to ameliorate the ill-effects of COVID-19 are disclosed. Non-limiting examples of ill-effects include fever, chills, cough, shortness of breath, difficulty breathing, fatigue, muscle ache, headache, loss of taste, loss of smell, sore throat, congestion, nausea, vomiting, diarrhea, pressure in chest, confusion, bluish lips, bluish face, secondary infection, pneumonia, sepsis, or death. In embodiments, ill-effects, other than death, are eliminated or reduced by 30 to 95%, such as 50% to 80%, or up to 100%. In embodiments, the frequency of death is reduced by 10 to 95%. In some embodiments, spike protein binding partner treatment results in the removal of SARS-CoV-2 from the bloodstream, or otherwise reduce blood levels.

In embodiments, spike protein binding partner is characterized as polypeptide configured to bind coronavirus spike protein. In some embodiments, a spike protein binding partner of the present disclosure includes or consists of one or more isolated polypeptides having a sequence identity to the mature polypeptide of SEQ ID NO: 1 of at least 60%, at least 70%, at least 80%, e.g., at least 85%, at least 90%, at least 91%, at least 92%, at least 93%, at least 94%, at least 95%, at least 96%, at least 97%, at least 98%, at least 99%, or 100%, having the ability to bind to the coronavirus spike protein. In some embodiments, a spike protein binding partner of the present disclosure includes or consists of one or more isolated polypeptides having a sequence identity to the mature polypeptide of SEQ ID NO: 2 of at least 60%, at least 70% e.g., at least 85%, at least 90%, at least 91%, at least 92%, at least 93%, at least 94%, at least 95%, at least 96%, at least 97%, at least 98%, at least 99%, or 100%, having the ability to bind to the coronavirus spike protein. In some embodiments, a spike protein binding partner of the present disclosure includes or consists of one or more isolated polypeptides having a sequence identity to the mature polypeptide of SEQ ID NO: 27 of at least 60%, at least 70% e.g., at least 85%, at least 90%, at least 91%, at least 92%, at least 93%, at least 94%, at least 95%, at least 96%, at least 97%, at least 98%, at least 99%, or 100%, having the ability to bind to the coronavirus spike protein. In embodiments, such spike protein binding partner may have one or more arginine amino acids attached thereto (such as 1-5, or 2 consecutive arginine amino acids), and/or one or more His-tags attached thereto (such as 3-7 consecutive histidine amino acids, or 6 consecutive amino acids).

In some embodiments, a spike protein binding partners of the present disclosure includes or consists of one or more polypeptides have identical subsequences to the corresponding subsequences in the 19-55 segment of ACE2 (SEQ ID NO. 1). In embodiments, polypeptides of the present disclosure comprise or consist of one or more polypeptides including residues corresponding to 19-55 of ACE2 (SEQ ID NO. 1), and further include (Arg)ₙ added to the carboxyl terminal end, wherein n is a number from 1 to 8. In embodiments, the one or more polypeptides may further include (His)ₙ added to the carboxyl terminal end, wherein n is a number from 1 to 8, or n=6.

In embodiments, polypeptides of the present disclosure comprise or consist of one or more polypeptides including residues corresponding to 22-45 of ACE2 (SEQ ID NO. 1), and further include (Arg)ₙ added to the carboxyl terminal end, wherein n is a number from 1 to 11, or wherein n is a number from 2 to 11. In embodiments, the one or more polypeptides may further include (His)ₙ added to the carboxyl terminal end, wherein n is a number from 1 to 8, or n=6. In embodiments, polypeptides of the present disclosure comprise or consist of one or more synthetic polypeptides including residues of SEQ ID NO: 27, and further include (Arg)ₙ added to the carboxyl terminal end, wherein n is a number from 1 to 11, or wherein n is a number from 2 to 11. In embodiments, the one or more polypeptides may further include (His)ₙ added to the carboxyl terminal end, wherein n is a number from 1 to 8, or n=6. In embodiments, polypeptides of the present disclosure comprise or consist of one or more synthetic polypeptides including residues of one of SEQ ID NOS. 2, 3 or 27, and further including (Arg)ₙ added to the carboxyl terminal end, wherein n is a number from 1 to 11, or wherein n is a number from 2 to 11, or wherein n is a number 2-8. In embodiments, the one or more polypeptides may further include (His)ₙ added to the carboxyl terminal end, wherein n is a number from 1 to 8, or n=6. In embodiments, polypeptides of the present disclosure comprise or consist of one or more synthetic polypeptides including residues of one of SEQ ID NOS. 2, 3 or 27, and further including (Xaa)ₙ added to the carboxyl terminal end, wherein in Xaa is selected from the group consisting of arginine, lysine, histidine, and combinations thereof, wherein n is a number from 1 to 11, or wherein n is a number from 2 to 11, or wherein n is a number 2-8. In some embodiments, n=0. In embodiments, the spike protein binding partners of the present disclosure have the ability to bind to the coronavirus spike protein. In embodiments, the spike protein binding partners of the present disclosure are able to decrease the binding ability of the coronavirus spike protein.

In some embodiments, a spike protein binding partner of the present disclosure includes or consists of an isolated polypeptide having a sequence identity to the polypeptide or mature polypeptide of SEQ ID NOS: 2-48 of at least 80%, e.g., at least 85%, at least 90%, at least 91%, at least 92%, at least 93%, at least 94%, at least 95%, at least 96%, at least 97%, at least 98%, at least 99%, or 100%, which have the ability to bind to the coronavirus spike protein. In embodiments, the isolated polypeptide is characterized as pharmaceutically acceptable.

In some embodiments, spike protein binding partner of the present disclosure includes one or more isolated polypeptides having a sequence identity to the mature polypeptide of SEQ ID NOS: 2-48 of at least 80%, e.g., at least 85%, at least 90%, at least 91%, at least 92%, at least 93%, at least 94%, at least 95%, at least 96%, at least 97%, at least 98%, at least 99%, or 100%, which have the ability to bind to the coronavirus spike protein. In embodiments, x or xaa is selected from the group consisting of arginine (R), Histidine (H), Lysine (K), and combinations thereof. In embodiments, x or xaa selected from the group consisting of arginine (R), Histidine (H), Lysine (K), include one or more positive charges sufficient to stabilize the α-helical conformation or a stabilized configuration.

In some embodiments, a spike protein binding partner of the present disclosure includes or consists of a polypeptide having the amino acid sequence consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48 having the ability to bind to the coronavirus spike protein. In embodiments, x or xaa is not any peptide, but rather is selected from the group consisting of Arginine (R), Histidine (H), Lysine (K), and combinations thereof. In some embodiments, the polypeptide is soluble in water at 25 degrees Celsius. In embodiments, the polypeptide is characterized as pharmaceutically acceptable, synthetic, substantially purified, or pure.

In some embodiments, spike protein binding partner of the present disclosure includes a plurality of polypeptides having amino acid sequences selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48. In embodiments, each amino acid sequence has the ability to bind to the coronavirus spike protein. In embodiments, x or xaa is not any amino acid, but rather is an amino acid selected from the group consisting of Arginine CR), Histidine (H), Lysine (K), and combinations thereof. In some embodiments, each polypeptide is soluble in water at 25 degrees Celsius. In embodiments, the polypeptide is characterized as isolated, or substantially purified.

In some embodiments, spike protein binding partner of the present disclosure includes a plurality of polypeptides having amino acid sequences selected from the group of amino acid sequences consisting of: SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26. In embodiments, each amino acid sequence has the ability to bind to the coronavirus spike protein. In embodiments, x or xaa is selected from the group consisting of Arginine (R), Histidine (H), Lysine (K), and combinations thereof. In some embodiments, each polypeptide is soluble in water at 25 degrees Celsius. In embodiments, the spike protein binding partner is characterized as isolated, or substantially purified.

In some embodiments, spike protein binding partner of the present disclosure includes a plurality of polypeptides having amino acid sequences selected from the group of amino acid sequences consisting of: SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26, wherein x, if present, is selected from the group consisting of Arginine (R), Histidine (H), Lysine (K), and combinations thereof. In some embodiments, each polypeptide is soluble in water at 25 degrees Celsius. In embodiments, the plurality of polypeptides are characterized as isolated, or substantially purified.

In some embodiments, spike protein binding partner of the present disclosure includes a plurality of polypeptides having amino acid sequences selected from the group of amino acid sequences consisting of: SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, and SEQ ID NO: 48, wherein x, if present, is selected from the group consisting of Arginine (R), Histidine (H), Lysine (K), and combinations thereof. In embodiments, X or xaa does not refer generally to any amino add. In some embodiments, each polypeptide is soluble in water at 25 degrees Celsius. In embodiments, the plurality of polypeptides are characterized as isolated, or substantially purified.

In some embodiments, suitable spike protein binding partner suitable for use in accordance with the present disclosure includes a recombinant variant of a parent angiotensin-converting enzyme 2 including: a plurality of deletions of amino acid residues corresponding to an amino acid residues M1 to 018 and E56 to F805, wherein SEQ ID NO:1 is used for numbering; and wherein the variant has increased stability, spike protein binding activity, and/or COVID-19 treatment performance benefit compared to the parent angiotensin-converting enzyme 2 or a reference angiotensin-converting enzyme 2 differing from the variant angiotensin-converting enzyme 2 only by the absence of the deletions. In embodiments, the variant has at least 90% amino acid sequence identity relative to SEQ ID NO: 1. In some embodiments, the variant angiotensin-converting enzyme 2 further includes one or more alterations of amino add residues including substitutions or insertions of one or more amino adds including a positive electrically charged side chain corresponding to an amino acid residues E56, E56 to E57, E56-N58, E56 to V59, E56 to Q60, E56 to N61, E56 to M62, or E56 to N63, using SEQ ID NO:1 for numbering, wherein the variant has increased stability, spike protein binding activity, and/or COVID-19 treatment performance compared to the parent angiotensin-converting enzyme 2 or a reference angiotensin-converting enzyme 2 differing from the variant angiotensin-converting enzyme 2 only by the absence of the deletions and absence of the one or more additions. Non-limiting examples of amino acid residues comprising a positive electrically charged side chain include arginine (R), Histidine (H), Lysine (K), and combinations thereof that stabilize the α-helical conformation. In some embodiments, the amino acid residues including a positive electrically charged side chain include at least two arginine residues such as two arginine residues, three arginine residues, four arginine residues, five arginine residues, six arginine residues, seven arginine residues, eight arginine residues, nine arginine residues, or more.

In some embodiments, alterations may be made to SEQ ID NO:1 such as deletions of amino acids 1-18 and one of 56 to 805, 57 to 805, 58 to 805, 50 to 805, 60 to 805, 61 to 805, 62 to 805 or 63 to 805, as well as at least one alteration corresponding to positions 56 to 63 using SEQ ID NO:1 for numbering. Non-limiting examples of alterations include one or more of the following insertions or substitutions: E56X, E57X, N58X, V59X, N61X, M62X, N63X, using SEQ ID NO:1 for numbering, wherein X is selected from one or more amino acid residues comprising a positive electrically charged side chain such as arginine (R), Histidine (H), Lysine (K), and combinations thereof.

In some embodiments, combinations of alterations include on or more of the following substitutions:

### E56X, E57X;

### E56X, E57X, N58X;

### E56X, E57X, N58X, V59X;

### E56X, E57X, N58X, V59X, Q60X;

### E56X, E57X, N58X, V59X, Q60 X, N61X:

### E56X, E57X, N58X, V59X, Q60X, N61X, M62X;

E56X, E57X, N58X, V59X, Q60X, N81X, M62X, N63X; using SEQ ID NO:1 for numbering, wherein X is selected from one or more amino acid residues comprising a positive electrically charged side chain such as arginine (R), Histidine (H), Lysine (K), and combinations thereof.

Non-limiting examples of alteration include one or more of the following alterations: E56R, E57R, N58R, V59R, Q60R, N61R, M62R, N63R using SEQ ID NO:1 for numbering.

In some embodiments, combinations of alterations include on or more of the following substitutions:

### E56R, E57R:

### E56R, E57R, N58R;

### E56R, E57R, N58R, V59R;

### E56R, E57R, N58R, V59R, Q60R;

### E56R, E57R, N58R, V59R, Q60R, N61R;

### E56R, E57R, N58R, V59R, Q60R, N61R, M62R;

E56R, E57R, N58R, V59R, Q60R, N61R, M62R, N63R; using SEQ ID NO:1 for numbering.

In some embodiments, the variant angiotensin-converting enzyme 2 further includes a plurality of deletions of amino acid residues corresponding to amino acid residues S19 to 121, and N49 to T55 using SEQ ID NO:1 for numbering. In some embodiments, the variants of the present disclosure further includes one or more alterations such as the addition or substitution of Arginine (R) amino acid residues corresponding to amino acid residues 149, 149 to Y50, N49 to N51, N49 to T52, N49 to N53, N49 to 154, N49 to T55, N49 to E56, using SEQ ID NO:1 for numbering, and wherein the variant has increased stability, spike protein binding activity, and/or COVID-19 treatment performance compared to the parent angiotensin-converting enzyme 2 or a reference angiotensin-converting enzyme 2 differing from the variant angiotensin-converting enzyme 2 only by the absence of the deletions and absence of the one or more additions.

In some embodiments, the present disclosure includes an isolated, pharmaceutically acceptable, and/or recombinant variant of a parent angiotensin-converting enzyme 2 comprising: a plurality of deletions of amino acid residues corresponding to amino acid residues M1 to I21 and S46 to F805 using SEQ ID NO:1 for numbering, and wherein the variant has increased stability, spike protein binding activity, and/or COVID-19 treatment performance benefit compared to the parent angiotensin-converting enzyme 2 or a reference angiotensin-converting enzyme 2 differing from the variant angiotensin-converting enzyme 2 only by the absence of the deletions. In some embodiments, the amino acid residues including a positive electrically charged side chain include at least two arginine residues such as two arginine residues, three arginine residues, four arginine residues, five arginine residues, six arginine residues, seven arginine residues, eight arginine residues, nine arginine residues, or more. In embodiments, the arginine residues are fused or afixed to the amino terminal end of the polypeptide. In some embodiments, the amino add sequences include (His)₆, or a His-Tag affixed to carboxy or amino terminal ends.

In embodiments, the parental angiotensin-converting enzyme 2 of the variants of the present disclosure is from Homo sapiens.

In some embodiments, the variant angiotensin-converting enzyme 2 of the present disclosure have at least 95% amino acid sequence identity to an amino acid sequence of at least one of SEQ ID NOS: 2-48. In some embodiments, the variant angiotensin-converting enzyme 2 of the present disclosure have at least 97% amino acid sequence identity to an amino acid sequence of at least one of SEQ ID NO: 2-48. In some embodiments, the variant angiotensin-converting enzyme 2 of the present disclosure has at least 99% amino acid sequence identity to one amino acid sequence of SEQ ID NO: 2-48. In some embodiments, the variant angiotensin-converting enzyme 2 of the present disclosure consists of one amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and combinations thereof. In some embodiments, each amino acid sequence is characterized as synthetic, pharmaceutically acceptable, substantially purified, or water soluble.

In some embodiments, the parental angiotensin-converting enzyme 2 amino acid sequence consists of SEQ ID NO: 1.

In embodiments, a recombinant variant of a parent angiotensin-converting enzyme 2 includes: a plurality of deletions of amino acid residues corresponding to an amino add residues M1 to 121 and N49 to F805 using SEQ ID NO:1 for numbering; and wherein the variant has increased stability, spike protein binding activity, and/or COVID-19 treatment. In embodiments, such variants include performance benefit compared to the parent angiotensin-converting enzyme 2 or a reference angiotensin-converting enzyme 2 differing from the variant angiotensin-converting enzyme 2 only by the absence of the deletions.

In embodiments, a recombinant variant of a parent angiotensin-converting enzyme 2 includes: a plurality of deletions of amino acid residues corresponding to an amino acid residues M1 to I21 and A46 to F805 using SEQ ID NO:1 for numbering; and wherein the variant has increased stability, spike protein binding activity, and/or COVID-19 treatment performance. In embodiments such variants include benefits compared to the parent angiotensin-converting enzyme 2 or a reference angiotensin-converting enzyme 2 differing from the variant angiotensin-converting enzyme 2 only by the absence of the deletions. In embodiments, a recombinant variant of a parent angiotensin-converting enzyme 2 includes: a plurality of deletions of amino acid residues corresponding to amino acid residues M1 to 121 and A46 to F805 using SEQ ID NO:1 for numbering; wherein the variant has spike protein binding activity. In embodiments, 1-8, 1-7, 1-6, 1-5, 1-4, 1-3, 1-2, or 1 arginine residues are added after residue L45 using SEQ ID NO:1 for numbering. In embodiments, 1-8, 1-7, 1-6, 1-5, 1-4, 1-3, 1-2, or 1 positive amino acid residues are added after residue L45 using SEQ ID NO:1 for numbering. In embodiments; 1-8, 1-7, 1-6, 1-5, 1-4, 1-3, 1-2, or 1 histidine residues are added after the arginine residues.

In embodiments, the variants of the present disclosure may include or further include a substitution, deletion, and/or insertion at one or more (e.g. several) positions. In embodiments, the number of amino acid substitutions, deletions, and/or insertions introduced into the mature polypeptide of SEQ ID NOS. 2-44 is up to 5 or up to 10, e.g. 1, 2, 3, 4, 5. In embodiments, the amino acid changes may be of a minor nature, that is conservative amino acid substitutions (such as 1-10 conservative substitutions) or insertions that do not significantly affect the folding and/or activity of the protein, small deletions, typically 1-3 amino acids.

In some embodiments, variants of the present disclosure may include a D-amino acid, e.g., D-alanine to the amino terminal end of the variant to prolong the half-life of the synthetic peptides in situ. In embodiments, a D-amino acid may be inserted into the peptide chain, or preferably exchanged, such that one of the existing amino acids may take the form of its optical isomer, i.e., the D-isomer, over the naturally occurring L-form. The D-amino acid may be part of the originally synthesized peptide. The D-amino acid may be synthetically placed on the synthetic peptides through the use of standard solid phase synthetic methods known to those skilled in the art. The conformation and helicity of the synthetic peptide with at least one D-amino acid added to the amino terminus of the parent peptide would not be expected to disrupt the active three-dimensional structure of the peptide. This can be confirmed in solution studies such as by Nuclear Magnetic Resonance (NMR).

In some embodiments, the present disclosure relates to a complementary deoxynucleotide (cDNA) sequence encoding an amino acid sequence having at least at least 90%, at least 95%, or at least 99% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, and SEQ ID NO: 48. In embodiments, the cDNA are made through laboratory manipulation and genetic engineering techniques. In embodiments, methods of making synthetic polypeptides of the present disclosure are known in the art.

In some embodiments, the present disclosure relates to a complementary deoxynucleotide (cDNA) sequence encoding an amino acid sequence selected from the group consisting of: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, and SEQ ID NO: 48.

In embodiments, the spike protein binding partners are purified according to pharmaceutically acceptable techniques and added to pharmaceutically acceptable composition or formulations such as dosage forms. In embodiments, the concentration of spike protein binding partners in a composition, such as an inhaler formulation, can vary a great deal, and will depend on a variety of factors, including the type and severity of one or more COVID-19 symptoms, the desired duration of relief from one or more COVID-19 symptoms, possible adverse reactions, the effectiveness of the spike protein binding partner(s), and other factors within the particular knowledge of the patient and physician. In certain embodiments, compositions of the present disclosure can include an amount of spike protein binding partner ranging from about 0.5 percent weight (wt %) to about 50 wt % of the total composition, in certain embodiments from about 0.5 wt % to about 5 wt % or the total composition, and in certain embodiments from about 5 wt % to about 20 wt % of the total composition. In embodiments, a single dosage may comprise about 2 to 3 micrograms/mL of one or more peptides in accordance with the present disclosure. In embodiments, a single about 2 to 3 micrograms/mL of one or more peptides in accordance with the present disclosure such as the polypeptides of SEQ ID NOS: 2-48, including SEQ ID NO: 39. In embodiments, the balance of the composition may include excipients, or adjuvants.

In embodiments, to prepare the drug product, the components of the pharmaceutical composition are blended and fabricated by methods known in the art. The resulting mixture is subsequently compacted in a press to yield individual (unit) dosages (blisters, tablets or capsules). To prepare the final drug product, the compressed dosage forms may undergo further processing, such as polishing, coating, and the like. In some embodiments, the spike protein binding partners are formulated into a pharmaceutically effective amount in a lyophilized formulation suitable for use in a pulmonary delivery device such as an inhaler. In embodiments, one or more spike protein binding partners are disposed within a pharmaceutically acceptable vehicle. In embodiments, a plurality of spike protein binding partners are administered via a pulmonary delivery device such as a dispenser. Accordingly, administration of the synthetic peptides of the present disclosure may be by oral, intravenous, intranasal, suppository, intraperitoneal, intramuscular, intradermal or subcutaneous administration or by infusion or implantation. When administered in such manner, the synthetic peptides of the present disclosure may be combined with other ingredients, such as carriers and/or adjuvants. There are no limitations on the nature of the other ingredients, except that they must be pharmaceutically acceptable, efficacious for their intended administration, cannot degrade the activity of the active ingredients of the compositions, and cannot impede importation of a subject peptide into a cell. The peptide compositions may also be impregnated into transdermal patches, or contained in subcutaneous inserts, such as in a liquid or semi-liquid form which patch or insert time-releases therapeutically effective amounts of one or more of the subject synthetic peptides. The pharmaceutical forms suitable for injection include sterile aqueous solutions or dispersions and sterile powders for the extemporaneous preparation of sterile injectable solutions or dispersions. The ultimate solution form in all cases must be sterile and fluid. Typical carriers include but are not limited to a solvent or dispersion medium containing, e.g., water buffered aqueous solutions, i.e., bio-compatible buffers, ethanol, polyols such as glycerol, propylene glycol, polyethylene glycol, suitable mixtures thereof, surfactants or vegetable oils. Sterilization may be accomplished utilizing any art-recognized technique, including but not limited to filtration or addition of antibacterial or antifungal agents. Examples of such agents include paraben, chlorbutanol, phenol, sorbic acid or thimerosal. Isotonic agents such as sugars or sodium chloride may also be incorporated into the subject compositions.

In embodiments, methods of treating or preventing one or more COVID-19 symptoms of the present disclosure can include administering to the subject a therapeutically effective amount of spike protein binding partner to a patient in need of such treatment. A spike protein binding partner, or a pharmaceutical composition containing same, can be administered orally such as through an inhaler, or intraperitoneally to the subject. Oral administration of spike protein binding partner to a subject includes administering an inhaled composition of the present disclosure such as a formulation for use in an inhaler. Non-limiting example if inhalers suitable for use herein include those described in U.S. Pat. No. 8,424,518 entitled Dry powder inhaler and system for drug delivery, as well as dry powder inhalers such as those described in U.S. Pat. Nos. 7,305,986 and 7,464,706, (all of which are herein incorporated by reference in their entirety). In embodiments, inhalers such as dry powder inhalers are configured to deliver medicaments such as a plurality of spike protein binding partners of the present disclosure to the lungs, and may contain a dose system of a powder formulation usually either in bulk supply or quantified into individual doses stored in unit dose compartments, like hard gelatin capsules or blister packs. In embodiments, bulk container are equipped with a measuring system operated by the patient in order to isolate a single dose from the medicaments such as powder immediately before inhalation. Dosing reproducibility requires that the drug formulation is uniform and that the dose can be delivered to the patient with consistent and reproducible results. Therefore, the dosing system ideally operates to completely discharge all of the formulation effectively during an inspiratory maneuver when the patient is taking his/her dose. However, complete discharge is not required as long as reproducible dosing can be achieved. Flow properties of the spike protein binding partner formulation, and long term physical and mechanical stability in this respect, may be more important for bulk containers than they are for single unit dose compartments. Good moisture protection can be achieved more easily for unit dose compartments such as one or more blisters. In embodiments, a unit dose inhaler is suitable for use in delivering a therapeutic effective amount of spike protein binding partner. In embodiments, a multiple dose inhaler is suitable or configured for delivering a therapeutic effective amount of spike protein binding partner of the present disclosure to a patient in need thereof.

Other suitable devices for pulmonary delivery of spike protein binding partners of the present disclosure to a patient in need thereof are described in International Publication Number WO2011/002406 entitled Dispenser and method for entraining powder in an airflow (herein entirely incorporate by reference). In embodiments, one dosage form suitable for administration of spike protein binding partners includes compositions such as a dry powder for inhalation. In embodiments, the amount of spike protein binding partner in a typical composition of the present disclosure can range from about 1 wt % to about 25 wt % of the total composition, such as about 5 wt % to 10 wt % of the total composition.

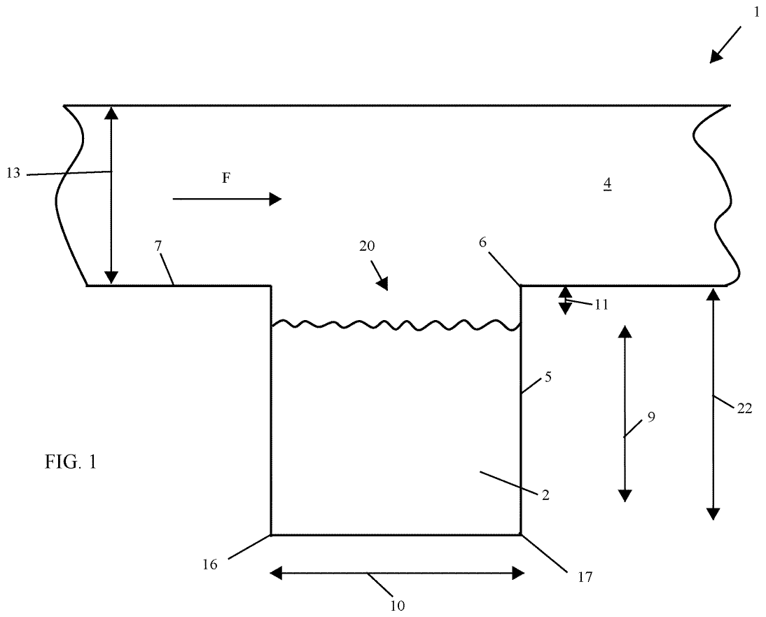

Referring now to FIG. 1, a cross-sectional side-view of a drug delivery device in accordance with the present disclosure is shown. In some embodiments, such as a pharmaceutical formulation for aerosolization, a lyophilized preparation, one or more spike protein binding partners of the present disclosure is dissolved in SWI buffer at pH 8 and atomized using vibrating mesh nebulizers Aeroneb Pro (Aerogen, Mountainview, Calif.) and EZ Breeze atomizer (Nephron Pharmaceuticals Co, Orlando, Fla.) as described (Surasarang S H et al, 2018, Formulation for a Novel Inhaled Peptide Therapeutic for Idiopathic Pulmonary Fibrosis Drug Dev Ind Pharm 44, 184-198).

Referring now to FIG. 1, device 1 includes a cavity 5 shown adjacent to flow path 4 of a first embodiment. The height of the flow path is shown at 13. In some embodiments, the cavity 5 may be a cuboid shaped and the cavity opening 20 has a rim 6 where the sides of the cavity 5 meet the flow passage lower wall 7. In embodiments, the cavity 5 includes medicament powder 2. In embodiments, medicament powder 2 includes or consists of one or more spike protein binding partners in accordance with the present disclosure alone or combination with excipients. In embodiments, cavity 5 is shaped to allow a cylindrical airflow pattern within the cavity 5. The cylindrical flow pattern in cavity 5 is developed around an axis located transverse to the flow direction and approximately in the middle of cavity 5. In embodiments, the sides of cavity 5 are perpendicular to lower wall 7.

In embodiments, device 1, configured for patient inhalation, includes a flow passage 4 adjacent to lower wall 7 (i.e. the lower wall of the passage when the device is in its normal operating orientation). In embodiments, the lower wall includes an opening 20 into the cavity 5 which may contain or hold a powder formulation and/or spike protein binding partners of the present disclosure. The passing of an air stream in the flow direction F along the flow passage and across the opening 20 generates a cylindrical circulating flow in cavity 5 due to a shear driven cavity flow. The medicament powder 2 including powder particles are agitated in this energetic, turbulent, circulating flow, and also impact the sides of cavity 5. In embodiments, the flow action contributes to deaggregation, bringing the medicament powder 2 into a condition ready for inhalation in passage 4.

In some embodiments, cavity 5 and cavity opening 20 each have a length 10 such as 1 to 10 mm in the flow direction F of the flow passage 4 of e.g., 5 mm. The cavity depth 22 is also about 5 mm. In embodiments, the distance 11 from the top of the cavity 5 (i.e. the plane of the cavity opening) to the top of the leveled powder particle bed in an initial condition is 1 mm. This distance is referred to as the headspace 11 of the cavity. The depth of medicament powder 2 such as a powder in the cavity 5 is shown at 9. In some embodiments, cavity 5 is square; the inner corners of cavity 5 are essentially sharp, e.g. the lower front (downstream) edge 16 and the lower rear (upstream) edge 17 are sharp. In a modification of the first embodiment (not shown), they may have a radius of about 0.5 mm in order to provide some guidance in the rotational movement of the generated circulating flow.

In embodiments, a powder-containing cavity 5 opens into a flow passage 4. The flow passage is configured to direct an inhalation air flow across the cavity opening. A circulating flow may be induced in the cavity 5 by shear driven cavity flow. In embodiments, powder is entrained in the circulating flow and deaggregared before exiting the cavity and becoming entrained in the flow of air along the flow passage 4 and toward the mouth of a user, such as when a patient inhales, in need of treatment in accordance with the present disclosure.

In some embodiments, an article of personal protection equipment, includes a plurality of spike protein binding partners disposed within or affixed upon one or more surfaces such as an inner layer, outer layer, or filter layer of an article of personal protection equipment. In embodiments, the personal protection equipment is configured for containment of or trapping SARS-CoV-2 when SARS-CoV-2 contacts the personal protection equipment. Non-limiting examples of suitable spike protein binding partners include those described herein above, including polypeptides having at least 90% sequence identity, at least 95% sequence identity, at least 99% sequence identity to SEQ ID NOS. 2-44. In embodiments, suitable spike protein binding partners for use in articles of the present disclosure include synthetic polypeptides including a plurality of polypeptides selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, and SEQ ID NO: 48, and combinations thereof. In embodiments, the spike protein binding partners are disposed within or affixed upon one or more surfaces of a respirator in a prophylactically effective amount of spike protein binding partner. In embodiments, a predetermined amount of spike protein binding partners are disposed within or affixed upon one or more surfaces of a respirator, wherein the respirator is configured to bind SARS-CoV-2 and/or variants of SARS-CoV-2.

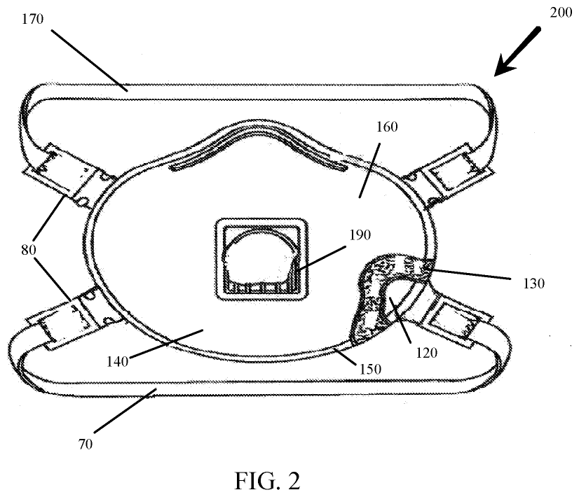

Referring now to FIG. 2, a partial cross-section frontal-view of a respirator in accordance with the present disclosure is shown. In embodiments, a cup-shaped disposable personal respirator 200 is shown in partial cross-section. Respirator 200 includes inner cover web 120, filtration layer 130, and outer cover layer 140. Welded edge 150 holds cover web 120, filtration layer 130, and outer cover layer 140 together and provides a face seal region to reduce leakage past the edge of respirator 200. Leakage may be further reduced by pliable dead-soft nose band 160 of for example a metal such as aluminum or a plastic such as polypropylene. Respirator 200 also includes one or more head and neck straps 170, and optional exhalation valve 190. Aside from the filtration layer 120, further details regarding the construction of respirator 200 will be familiar to those skilled in the art and embodiments may include respirators such as those described in U.S. Pat. Nos. 7,905,973, 10,661,105, 10,130,833, 9,468,872 all of which are entirely incorporated by reference. In embodiments, one or more of inner cover web 120, filtration layer 130, and outer cover layer 140 includes a plurality of spike protein binding partners in a prophylactically effective amount. In embodiments, the spike protein binding partners are bound to inner cover web 120, filtration layer 130, and/or outer cover layer 140 by an adhesive tie layer. In embodiments, spike protein binding partners are disposed and or affixed atop the inner surface, i.e. the inner most surface adjacent to the skin of a user. In embodiments, spike protein binding partners are disposed and or affixed atop the outer surface of the outer cover layer 140. In embodiments, the spike protein binding partners are disposed and/or affixed within the filtration layer 130.

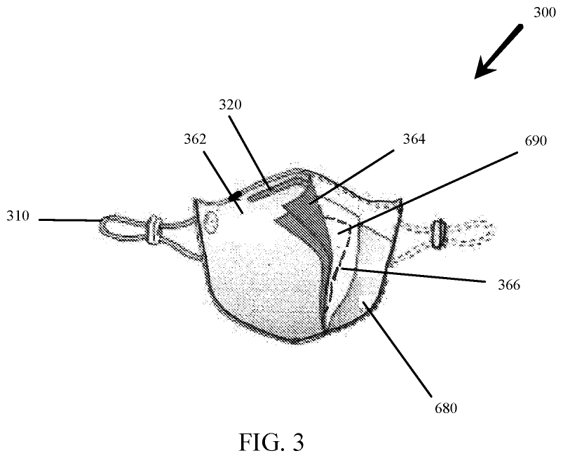

Other masks that may be modified in accordance with the present disclosure include N95 respirators such as Model: Professional BF-200-3013 An available as BIOFRIEND™ BIOMASK™ brand N95 surgical respirator manufactured by Filligent (HK) Limited. Referring now to FIG. 3 a partial cross-section frontal-view of a respirator in accordance with the present disclosure is shown. In embodiments, the respirator 300 is configured to be flatfolded and to expand into a convex-shaped mask with polyamide/spandex elastic head-loops 310 to secure the mask to the user's face. In embodiments, respirator 300 includes malleable strip 320 such as an aluminum strip positioned above the nose of a user for a tight seal around the nose and face of the user. In some embodiments, the respirator 300 may include several layers of material, such as three or more layers. As shown in FIG. 3 five layers of material include: an outer layer 362 such as a spun-bond polypropylene, a second layer 364 of cellulose/polyester, a third layer 366 of melt-blown polypropylene filter material and an inner 680 (fourth) layer of spun-bound polypropylene. In embodiments an antiviral layer 690 is positioned between second layer 364 and third layer 366. In embodiments, the construction materials used herein include typical construction materials commonly used in surgical facemasks and N95 surgical respirators. In some embodiments, outer layer 362 of the respirator 300 is coated with a hydrophilic plastic. In some embodiments, the second layer 364 is treated with one or more metal ions. In embodiments, all layers may be configured to inactivate SARS-CoV-2 by incorporating, infusing or bonding a plurality of spike protein binding partners in accordance with the present disclosure. In some embodiments, an optional fifth layer such as antiviral layer 690 including cotton may be incorporated between one or more of the first, second, third, or fourth layers (between second layer 364 and third layer 366 as shown in FIG. 3). The cotton may include spike protein binding partners of the present disclosure to bind, immobilize and/or trap SARS-CoV-2.

In embodiments, the antiviral layer 690 comprises or consisting of cotton impregnated with one or more spike protein binding partners in accordance with the present disclosure such as in a prophylactically effective amount. Covalent linkage of peptides to cotton has been established (see e.g. Orlandin, A.; Dolcet, P.; Biondi, B.; Hilma, G.; Coman, D.; Oancea, S.; Formaggio, F.; Peggion, C. Covalent Graft of Lipopeptides and Peptide Dendrimers to Cellulose Fibers. Coatings 2019, 9, 606), and essentially uses conventional solid phase peptide synthesis procedures. In embodiments, cotton is treated with Fmoc-NHCH2CH2-NH2, following Fmoc removal, peptides of the present disclosure such as spike protein binding partners are synthesized by incorporating one amino acid at a time, beginning with the formation of an amide bond with the free NH2 of 1,2-diaminoethane, until completion of the entire polypeptide and removal of blocking groups.

In some embodiments, the present disclosure relates to an article of personal protection equipment, including: a plurality of polypeptides selected from the group consisting of SEQ ID NOS.: 2-26, SEQ ID NOS.:4-26, SEQ ID NO: 27-44, or combinations thereof disposed within or upon the surface of the personal protection equipment, wherein the personal protection equipment is configured to contain SARS-CoV-2 (or variants thereof) when SARS-CoV-2 contacts the personal protection equipment. In embodiments, the plurality of polypeptides are present in a prophylactically effective amount.

In embodiments, the present disclosure provides designed peptides such as spike protein binding partners described herein that bind very specifically to the Covid-19 virus. The spike protein binding partners can bind to the virus, preventing viral binding to receptors on cells that enable its entry into the cells, that results in cell death and disease. The spike protein binding partners can be used to block the virus from entering the respiratory system by their attachment to face filtering devices, and by delivering them as aerosols to the nasal cavities and upper respiratory tract where they can bind to air and droplet-borne viruses preventing their entry into cells.

In some embodiments, the spike protein binding partners of the present disclose are subjected to molecular tagging methods, or the expression or recombinant proteins as fusion molecules with histidine tags which typically include three to six consecutive histidine residues. The resultant proteins, containing the histidine residues have useful properties such as the ability to chelate the free coordination sites of metal ions. Their ability is conferred by the imidazole moieties of the histidines.

Expression vectors designed for the production of histidine tag fusion proteins are commercially available. These vectors encode a histidine tag up-stream or down-stream of the cloning site. Alternatively, the histidine tag may be fused to the gene of interest (such as a cDNA of the present disclosure) by polymerase chain reaction (PCR), for example by including the histidine tag sequence in the primers used for the amplification of the DNA of interest. In embodiments, PCR amplified DNA fusion product may be cloned into an expression vector comprising appropriate regulatory signals for transcription and translation. The tagged protein/s of interest can be expressed in suitable host cells which have been transfected, transformed or infected with the appropriate expression vector carrying the DNA sequence of the histidine tagged protein/s of interest and cultured. The recombinant proteins produced can be soluble, insoluble (inclusion bodies), intracellular, periplasmic or secreted to the medium. His-Tags and use thereof is further described in U.S. Pat. Nos. 6,869,775, and 6,416,959, and EP Patent Publication no. 0991944.

In some embodiments, the present disclosure relates to a bead including: a substrate; and one or more spike protein binding partners. For example, referring to FIG. 3, a bead composition 100 is shown including substrate 105, and one or more spike protein binding partners 110. In some embodiments, the one or more spike protein binding partners 110 include one or more polypeptides with SARS-CoV-2 spike protein binding affinity fixedly attached to the substrate 105. In some embodiments, the one or more polypeptides with SARS-CoV-2 spike protein binding affinity fixedly attached to the substrate is one or more spike protein binding partners of the present disclosure, or one or more fragments thereof. In some embodiments, the one or more polypeptides with SARS-CoV-2 spike protein binding affinity fixedly attached to the substrate is one or more spike protein binding partners of the present disclosure including a His-Tag fixedly attached or fused to the N-terminal or C-terminal end of the one or more spike protein binding partners, or one or more fragments thereof. In some embodiments, the one or more polypeptides with spike protein binding affinity are fixedly attached to the substrate incudes a synthetic amino acid sequence, comprising, or consisting of SEQ ID NOS: SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48. In embodiments, the amino acid sequences include a His-Tag. In embodiments, the substrate is a metal such a nickel, and the His-Tag binds to the substrate.

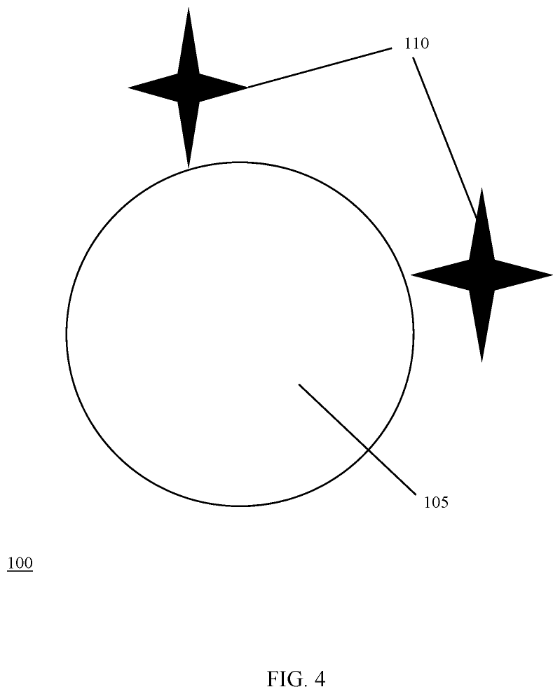

Still referring to FIG. 4, in some embodiments, the substrate 105 includes, one or more magnetic beads, nickel beads, nanobeads made of or including nickel, or combinations thereof. In embodiments, the beads are characterized as nanobeads.

In embodiments, a His-Tag may be fused to the polypeptides of the present disclosure using methods known in the art. For example, a cDNA sequence, genomic DNA, or a novel gene or EST with open reading frames encoding a polypeptide of the present disclosure may be fused to a DNA sequence encoding one or more histidine tags, which may include four or more (e.g., 4, 5, 6, 7, 8, 9) consecutive histidine residues. In embodiments, the tag can be fused to the N-terminal or C-terminal end of the encoded polypeptide of interest. Alternatively, the tag can be placed within the coding region of the gene, provided that it does not affect the activity of the protein. Alternatively, an endogenous-natural stretch of histidine residues can be used as a tag as well. In embodiments, histidine-tagged proteins itself may be used to explore the therapeutic value of the novel protein and its mechanism of action. In embodiments, a histidine-tag includes or consists of 6 consecutive histidines.

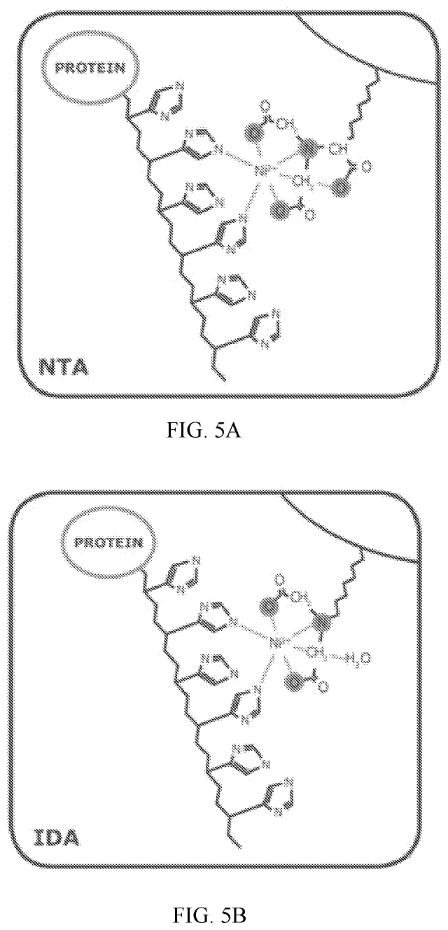

Referring now to FIGS. 5A and 5B, embodiments of the present disclosure include a hexa-histidyl sequence on the carboxyl terminal end of the 22-45 ACE2 peptide (“protein” in the figure) attached to nano beads (upper right in FIGS. 5A and 5B) that chelate nickel with chelating moieties attached to the beads. NTA is nitrilotriacetic acid chelator (FIG. 5A), IDA is iminidiacetic acid (FIG. 5B).

In embodiments, the present disclosure includes a bead including: a substrate; and one or more spike protein binding partners. In embodiments, the one or more spike protein binding partners include a His-Tag, wherein the His-Tag is bound to the substrate. In embodiments, the substrate comprises metal, such as nickel. In embodiments, the one or more spike protein binding partner include one or more polypeptides with SARS-CoV-2 spike protein binding affinity fixedly attached to the substrate. In embodiments, the one or more polypeptides with SARS-CoV-2 spike protein binding affinity fixedly attached to the substrate is one or more spike protein binding partners or one or more fragments thereof. In embodiments, the substrate is a magnetic particle. In embodiments, the substrate is a spherical magnetic particle.

It should be understood that the amino acid sequences such as spike protein binding partners of the present disclosure may be synthesized using methods known in the art for making polypeptide sequence.

### EXAMPLES

### Structure of the ACE2 α1 Domain.

The helical probability profiles for isolated 19-55 ACE2 peptide were computed using four different programs based on different approaches and which utilize different data bases. The results of employing multiple approaches uniformly show that the peptide has high α-helical content. As shown in Table 1, sequences from residues 22-47 are predicted to be almost entirely α-helical by all methods. Experimentally, the residues that make direct contact with the RBD of the SARS-CoV-2 spike protein are Gln 24-Gln 42, all of which are included in the 22-45 subdomain predicted to be α-helical by all of the different methods. Therefore, the 19-55 peptide contains the critical subdomain that binds to the spike protein, and is expected to do so when adopting its native binding conformation.

Two peptides from ACE2 and two corresponding modified ACE2 peptides are synthesized using solid phase methods and the peptides interfere with the coronavirus' binding to the ACE2 receptor protein as follows:

Amino acid sequences consisting of SEQ ID NO:2, SEQ ID NO:3, SEQ ID NO:4 and SEQ ID NO: 5. The peptides of SEQ ID NOS.: 4 and 5 are the same as peptides of SEQ ID NOS.: 2 and 3, respectively, except that a set of positively charged amino acid residues have been added, i.e., (Arg)₈. The present disclosure incorporates this addition because positive charges on the carboxyl terminal end of an α-helix tend to stabilize this structure. Also negatively charged amino acid residues on the amino terminal end of an α-helix likewise stabilize this structure. In embodiments, 1-3 negatively charged amino acid residues may be added to the amino terminal end of the spike protein binding partners described above. Fortuitously, the ACE2 peptide has 2 Glu (negatively charged —COO⁻ side chains) residues at the amino terminal ends of these peptides, already resulting in α-helical structure stabilization. Using several different secondary structure prediction algorithms it was found that addition of Arg residues to the carboxyl terminal of the 22-45 peptide (using SEQ ID NO: 1 for numbering) resulted in increased alpha-helical probability of this peptide. Addition of six His residues results in an even higher alpha helical probability for this segment.

### Inhibition of Viral Entry by Isolated Peptides.

Established methodologies are used which are commonly employed by typical practitioners in the field of virology.

Peptides of SEQ ID NO:2, SEQ ID NO:3, SEQ ID NO:4 and SEQ ID NO: 5 Used to Block SARS-CoV-2 Viral Entry into Cells.

Each of the peptides of SEQ ID NO:2, SEQ ID NO:3, SEQ ID NO:4 and SEQ ID NO: 5 is used in SARS-CoV-2 Covid-19 viral neutralization assays.

1. In one assay, inactive virus or virus expressing the SARS-CoV-2 spike protein and which contains a luciferase encoded gene is incubated with cells expressing the transmembrane ACE2 receptor either in the absence (control) or presence of different concentrations of each of the four above-listed peptides. The cells are then tested for luciferase expression. The lower expression of luciferase induced by the peptides indicates inhibition of viral penetration of the cells.

Viral neutralization is measured in a standard S protein expressing lentiviral cell infectivity assay. In this assay, SARS-CoV-2 spike pseudotyped lentiviruses expressing a luciferase reporter gene are produced by transfecting 293T cells with three plasmids: approx 7 ug of pCMVDR8.2, 7 ug of pHROCMV-Luc (luciferase-expressing plasmid) and 400 ng CMV/Covid-S. Cells are transfected overnight, washed and replenished with fresh media. Forty-eight hours later, supernatants are harvested and filtered. To confirm viral expression, the supernatants are assayed for expression of p24 using the standard Coulter HIV-1 p24 Antigen Assay kit (Beckman Coulter, Brea, Calif., USA). Aliquots of these supernatants are then added to Vero cells in 48-well dishes (approx 30,000 per well) with each peptides over a concentration range of 0-200 ug/mL. After incubation of the cells with virus+peptide, the cells are lysed in mammalian cell lysis buffer (Promega) and subjected to the luciferase assay using the Promega reagent.

2. In an alternate assay, BHK-21 cells (1.6×10⁵ cells/mL) are transfected with ACE2 and DsRed as a negative control. After 24 h, cells are washed with PBS and infected with 8×10⁷ genome equivalents (GE) per 24-well of SARS-CoV-2 isolate for 1 h at 37 degrees C. Calu-3 cells (5×10⁵ cells/mL) are mock-treated or treated with 100 mM camostat mesylate (Sigma Aldrich, St. Louis, Mo. USA) 2 h prior to infection with SARS-CoV-2 isolate at a multiplicity of infection (MOI) of 0.001 for 1 h at 37 degrees C. After infection, cells are washed three times with PBS before 500 ml of DMEM medium is added. At 16 or 24 h post infection, 50 ml culture supernatant is subjected to viral RNA extraction using a viral RNA kit (Macherey-Nagel) according to the manufacturer's instructions. GE per ml are detected RTPCR.

TABLE 1

| | | SEQUENCE NOS |
|---|---|---|
| | OVERALL | FOR α-HELIX |
| | α-HELIX | PROBABILITY |
| METHOD | PROBABILITY (%) | >70% |
| Chou-Fasman (Ref. 5) | 68 | 21-45 |
| JNET (Ref. 6) | 70 | 22-48 |
| JHMM (Ref. 6) | 81 | 21-51 |
| JPSSM (Ref 6) | 68 | 22-47 |

### Example 2

Further strong support that the ACE2 alpha 1 peptide binds to the receptor binding domain (RBD) of Covid-19 is rendered from molecular dynamics simulations of the structure of residues 22-45 of ACE2 bound to residues 319-541. The recently determined x-ray structure of this complex (PDB Structure 6VW1; Shang J et al [2020] Structural basis of receptor recognition by SARS-CoV-2 Nature doi.org/10.1038/s41586-020-2179-y) was used as the starting conformation for energy minimization of the complex followed by 10 nsec molecular dynamics calculations using the Amber forcefield with explicit water molecules. The average structure was close to the energy-minimized starting conformation. Importantly, the structure of the 22-45 ACE2 peptide was an alpha-helix that was superimposable of the structure of the energy-minimized alpha 1 starting structure (RMSD<1 Å). There was a small deviation of residues 43-45. Further molecular dynamics studies were performed on the 22-45 ACE2 segment which contained two positively charged Arg residues that would stabilize the alpha-helical structure on the carboxyl terminal end of the sequence (see below). The resulting average molecular dynamics structure was again an alpha-helix that was closely superimposable on the energy-minimized starting structure, now including residues 43-45 that adopted an alpha-helical conformation that was superimposable on the corresponding residues of the energy-minimized starting structure. Unexpectedly and significantly, the two extra Arg residues disclosed in this invention, made a series of favorable contacts with residues on the corona viral RBD, including Asp 420, Tyr 421, the backbone C═O of Arg 457 and Try 473. This result suggests that ACE2-22-45-Arg-Arg binds with an enhanced affinity over the un-arginylated peptide.

### Prophetic Example 3

A 65-year-old male with comorbidities presents with one or more COVID-19 symptoms. The patient is administered a therapeutically effective amount of a spike protein binding partner of the present disclosure. The COVID-19 symptoms subside, and lung function is improved.

### Prophetic Example 4

An ER doctor, in close proximity to a subject infected with SARS-CoV-2 virus, dons a mask in accordance with the present disclosure. The mask binds aerosolized SARS-CoV-2 virus, and the doctor avoids infection.

### Prophetic Example 5

A 65-year-old male with comorbidities presents with one or more COVID-19 symptoms. The patient is administered a therapeutically effective amount of a spike protein binding partner of including amino acids 22-45 using SEQ ID. NO 1 for numbering. The COVID-19 symptoms subside, and lung function is improved.

### Prophetic Example 6

A 65-year-old male with comorbidities presents with one or more COVID-19 symptoms. The patient is administered a therapeutically effective amount of a spike protein binding partner of including, or consisting of, amino acids 22-45 using SEQ ID. NO 1 for numbering, with the addition of two arginine residues on the C-terminal end. The COVID-19 symptoms subside, and lung function is improved.

The entire disclosure of all applications, patents, and publications cited herein are herein incorporated by reference in their entirety.

1. A recombinant variant of a parent angiotensin-converting enzyme 2 comprising: a plurality of deletions of amino acid residues corresponding to amino acid residues M1 to Q18 and E56 to F805 using SEQ ID NO:1 for numbering, and wherein the variant has increased stability, spike protein binding activity, and/or COVID-19 treatment performance benefit compared to the parent angiotensin-converting enzyme 2 or a reference angiotensin-converting enzyme 2 differing from the variant angiotensin-converting enzyme 2 only by an absence of the deletions.

2. The variant angiotensin-converting enzyme 2 of claim 1, wherein the variant further comprises one or more additions of amino acid residues comprising one or more positive electrically charged side chain corresponding to amino acid residues E56, E56 to E57, E56-N58, E56 to V59, E56 to 060, E56 to N61, E56 to M62, or E56 to N63, using SEQ ID NO:1 for numbering, and wherein the variant has increased stability, spike protein binding activity, and/or COVID-19 treatment performance compared to the parent angiotensin-converting enzyme 2 or a reference angiotensin-converting enzyme 2 differing from the variant angiotensin-converting enzyme 2 only by the absence of the deletions and absence of the one or more additions.

3. The variant of claim 2, wherein the amino acid residues comprising one or more positive electrically charged side chain comprise arginine, histidine, lysine, or combinations thereof.

4. The variant of claim 2, wherein the amino acid residues comprising one or more positive electrically charged side chain comprise at least two arginine residues.

5. The variant angiotensin-converting enzyme 2 of claim 1, wherein the variant further comprises a plurality of deletions of amino acid residues corresponding to the amino acid residues S19 to I21, and N49 to T55.

6. The variant angiotensin-converting enzyme 2 of claim 5, wherein the variant further comprises one or more additions of arginine (R) amino acid residues corresponding to amino acid residues N49, N49 to Y50, N49 to N51, N49 to T52, N49 to N53, N49 to 154, N49 to T55, N49 to E56, using SEQ ID NO:1 for numbering, and wherein the variant has increased stability, spike protein binding activity, and/or COVID-19 treatment performance compared to the parent angiotensin-converting enzyme 2 or a reference angiotensin-converting enzyme 2 differing from the variant angiotensin-converting enzyme 2 only by the absence of the deletions and absence of the one or more additions.

7. The variant angiotensin-converting enzyme 2 of claim 1, wherein the parental angiotensin-converting enzyme 2 is from Homo sapiens.

8. The variant angiotensin-converting enzyme 2 of claim 1, wherein the parental angiotensin-converting enzyme 2 or the variant has at least 95% amino acid sequence identity to an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48.

9. The variant angiotensin-converting enzyme 2 of claim 1, the variant has at least 97% amino acid sequence identity to an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48.

10. The variant angiotensin-converting enzyme 2 of claim 1, the variant has at least 99% amino acid sequence identity to an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48.

11. The variant angiotensin-converting enzyme 2 of claim 1, wherein the parental angiotensin-converting enzyme 2 amino comprises an amino acid sequence consisting of SEQ ID NO: 1.

12. The variant angiotensin-converting enzyme 2 of claim 1, wherein the variant consists of amino acids selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, and SEQ ID NO: 44.

13. The variant angiotensin-converting enzyme 2 of claim 1, wherein the variant comprises an amino acid sequence consisting of SEQ ID NO:2, SEQ ID NO: 3, or SEQ ID NO: 27.

14. A synthetic amino acid sequence, comprising, or consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, or SEQ ID NO: 48.

15. A synthetic amino acid sequence of claim 14, further comprising: a His-tag.

16. A pharmaceutically acceptable composition comprising a variant angiotensin-converting enzyme 2, or an amino acid sequence of claim 1.

17. The composition of claim 16, wherein the composition is effective for treating, ameliorating, or preventing COVID-19.

18. A method of treating, ameliorating, or preventing one or more symptoms of COVID-19 in a subject having one or more COVID-19 symptoms, comprising: administering a therapeutically effective amount of a spike protein binding partner to a subject in need thereof.

19. The method of claim 18, wherein the spike protein binding partner has a binding affinity sufficient to bind to SARS-CoV-2 spike protein.

20. The method of claim 18, wherein the spike protein binding partner binds one or more SARS-CoV-2 spike proteins.

21-37. (canceled)
